(19) United States
(12) Patent Application Publication
(10) Pub. No.: US 2025/0025535 A1
(43) Pub. Date: Jan. 23, 2025

(54) A COMPOSITION COMPRISING NANOSIZED ACTIVE PHARMACEUTICAL INGREDIENT

(71) Applicant: NANOFORM FINLAND OYJ, Helsinki (FI)

(72) Inventors: Panu NOPPARI, Helsinki (FI); Pia RUNEBERG-ROOS, Helsinki (FI); Barry DERHAM, Helsinki (FI); Maria LUME, Helsinki (FI); Satu LAKIO, Helsinki (FI)

(21) Appl. No.: 18/710,561

(22) PCT Filed: Nov. 17, 2022
(86) PCT No.: PCT/FI2022/050759

§ 371 Date: May 15, 2024

(30) Foreign Application Priority Data
Nov. 19, 2021 (FI) 20216192

(51) Int. Cl. A61K 38/29; A61K 9/51; A61K 38/22; A61K 38/23; A61K 38/28; A61K 47/44

(52) U.S. Cl. CPC A61K 38/29; A61K 9/51; A61K 38/22; A61K 38/23; A61K 38/28; A61K 47/44

(57) ABSTRACT

The present disclosure relates to compositions including suspensions comprising one or more nanosized active pharmaceutical ingredients (APIs) and one or more pharmaceutically acceptable oils, wherein the APIs are selected from proteins and peptides. When the APIs were nanosized and suspended in a pharmaceutically acceptable oil, their release to aqueous environment (i.e., recovery) was hindered compared to the corresponding bulk APIs suspended in the pharmaceutically acceptable oil.

### FIELD

The present disclosure relates to compositions comprising nanosized active pharmaceutical ingredients, in particular to compositions comprising nanosized active pharmaceutical ingredients suspended in pharmaceutically acceptable oils. The disclosure also relates to the compositions for use as medicaments.

### BACKGROUND

The oral route of drug administration is preferred over injections due to its ease of administration, high patient compliance, and low manufacturing costs. However, due to various gastrointestinal barriers to drug absorption, the oral route is often unsuitable for delivery of several drugs such as biologics.

Sharifi-Rada et al (J. of Biomolecular Structure and Dynamics, 2021, vol 39, no 3, pp 1029-1043) disclose oil-in-water nano emulsions comprising Berberine in olive oil to improve its aqueous-phase solubility.

Jeong et al (Pharmaceutics 2021, 13, 1050) disclose that clearance and bioavailability of methotrexate-loaded nano emulsions tend to decrease by 99% and increase by 19%, respectively, compared to those of the nanoparticles.

CN1456351A discloses an oral-applied self-emulsifying polypeptide drug formulation which comprises the polypeptide, gel adhesive surfactant, co-surfactant, oil, enzyme inhibitor, and a diluent. When the drug formulation reaches the intestinal tract, it is emulsified by itself to become a microemulsion.

CN106334185B discloses an oral composition containing nano emulsion of a polypeptide drug which is prepared by mixing the polypeptide with co-surfactant, thereafter the mixture is added into an oil phase. Next, a surfactant is added to the mixture in the oil phase, giving rise to a nano emulsion. The nano emulsion obtained was encapsulated in an enteric soft capsule.

However, there is still need for further compositions of APIs suitable for oral administration.

### SUMMARY

It was observed that bioavailability, release, distribution, and/or stability of certain active pharmaceutical ingredients (APIs) could be enhanced when the APIs are nanosized and suspended into a pharmaceutically acceptable oil.

Accordingly, it is an object of the present disclosure to provide composition including a suspension comprising
one or more nanosized active pharmaceutical ingredients (APIs) selected from proteins and peptides, and
one or more pharmaceutically acceptable oils.

It is also an object of the present disclosure to provide a composition of claim 1 for use as a medicament.

A number of exemplifying and non-limiting embodiments of the invention are described in accompanied dependent claims.

Various exemplifying and non-limiting embodiments of the invention and methods of operation, together with additional objects and advantages thereof, are best understood from the following description of specific exemplifying embodiments.

The verbs “to comprise” and “to include” are used in this document as open limitations that neither exclude nor require the existence of also unrecited features.

The features recited in dependent claims are mutually freely combinable unless otherwise explicitly stated. Furthermore, it is to be understood that the use of “a” or “an”, i.e., a singular form, throughout this document does not exclude a plurality.

### BRIEF DESCRIPTION OF FIGURES

FIG. 1 shows SEM images of bulk HSA (A) and nanosized HSA (B), scale bars 1 μm.

FIG. 2 shows SEM images of bulk insulin (A) and nanosized insulin (B), scale bars 1 μm.

FIG. 3 shows SEM images of bulk lysozyme (A), microsized lysozyme (B) and nanosized lysozyme (C), scale bars 1 μm.

FIG. 4 shows SEM images of bulk D-mannitol (A) and nanosized D-mannitol (B), scale bars 1 μm.

FIG. 5 shows sedimentation of nanosized insulin (left tube in each panel) and bulk insulin (right tube in each panel) in fish oil as a function of time. A: t=0 min; B: t=30 min; C: t=60 min; D: t=24 h.

FIG. 6 shows extraction recovery (% of theoretical total) of bulk HSA (black bar) and nanosized HSA (white bar) from fish oil.

FIG. 7 shows extraction recovery (% of theoretical total) of insulin (black bar) and nanosized insulin (white bar) from olive oil and fish oil.

FIG. 8 shows extraction recovery (% of theoretical total) of lysozyme (black bar) and nanosized lysozyme (white bar) from fish oil.

FIG. 9 shows extraction recovery (% of theoretical total) of D-mannitol (black bar) and nanosized D-mannitol (white bar) from olive oil.

FIG. 10 shows extraction recovery (% of theoretical total) of bulk lysozyme (black bar) microsized lysozyme (checkered bar) and nanosized lysozyme (white bar) from fish oil.

FIG. 11 shows RP-HPLC chromatograms of 10 mg bulk insulin suspended in 1 mL fish oil mixed for 1 h at 37° C. in 50 mL aqueous buffer in the presence of chymotrypsin (top panel); and 10 mg nanosized insulin suspended in 1 mL oil mixed for 1 h at 37° C. in 50 mL aqueous buffer in the presence of chymotrypsin (bottom panel). (1=background peak from oil; 2=insulin degradation product; 3=intact insulin; 4=PMSF proteinase added to the mixture inhibitor after the reaction). Samples were taken from the aqueous phase.

### DESCRIPTION

The specific examples provided in the description given below should not be construed as limiting the scope and/or the applicability of the appended claims. Lists and groups of examples provided in the description given below are not exhaustive unless otherwise explicitly stated.

As defined herein a nanosized API consists of particles which Dv90 is equal to or is less than 900 nm, i.e., a nanosized API consist of particles which 90% of volume fraction has a diameter below 900 nm. The API particle size may be between 10 nm and 900 nm, for example between 10 nm and 200 nm, between 200 nm and 500 nm, or between 500 nm and 900 nm. The size distribution can be tuned as desired since the preferred size of the nanoparticles may be API and application dependent.

As defined herein, a suspension is a heterogenous mixture of a fluid which contains solid particles dispersed in a continuous liquid phase. The solid particles (here the API particles) are not dissolved in the liquid phase (here pharmaceutically acceptable oil). The terms suspension and dispersion are used in the present disclosure as synonyms.

As defined herein, an emulsion is a heterogenous fluid consisting of at least two separate liquid phases; a dispersed liquid phase in a continuous liquid phase. A nanoemulsion is simply an emulsion where the dispersed liquid phase is nanosized within the continuous liquid phase. It is important to distinguish that emulsions do not contain a solid phase (i.e., solid particles). The suspensions of the present disclosure are not emulsions or nanoemulsion as defined herein.

The present disclosure is based on the observation that when certain active pharmaceutical ingredients (APIs) were nanosized and suspended in a pharmaceutically acceptable oil, their release to aqueous environment (i.e., recovery) was hindered compared to the corresponding bulk APIs suspended in the pharmaceutically acceptable oil. This finding implies that the released amount as well as the release rate of the nanosized API is slower compared to the corresponding bulk API. Furthermore, sedimentation rate of the nanosized API in the pharmaceutically acceptable oils was slower compared to the corresponding bulk API implicating that the nanosized API is more readily dispersed. This makes the nanosized API easier to handle and to aliquot by pipetting as a suspension in oil during manufacturing. A more homogenous dispersion can provide improved content uniformity in dosage forms, a more controlled and potentially even a sustained release of the API. As handling and weighing of dry nanosized API is time consuming, pipetting of suspensions of nanosized API in oil can be a mean to achieve more efficient manufacturing routines. These findings allow for the development of compositions comprising nanosized APIs and pharmaceutically acceptable oils and their use as medicaments.

According to one aspect, the present disclosure relates to a composition including nanosized particles of one or more APIs suspended in one or more pharmaceutically acceptable oils, also known as a suspension comprising the APIs and the pharmaceutically acceptable oil. The APIs of the present disclosure are selected from proteins and peptides.

According to one embodiment, composition consists of a suspension comprising one or more APIs suspended in one or more pharmaceutically acceptable oils.

According to another embodiment, composition consists of a suspension consisting of one or more APIs suspended in one or more pharmaceutically acceptable oils.

According to a particular embodiment, the API is selected from insulin, parathyroid hormone, oxytocin, calcitonin, vasopressin, and human serum albumin, preferably insulin.

According to one embodiment a protein is an antibody.

According to one embodiment, molecular weight of the API is 500 kDa or less.

According to one embodiment, the molecular weight of the API is 250 kDa or less.

The suspension can include more than one API provided that at least one of the APIs, preferably all APIs are nanosized.

The pharmaceutically acceptable oil can be any oil suitable for use in pharmaceutical compositions. Exemplary non-limiting pharmaceutically acceptable oils suitable for the present disclosure are selected from a group consisting of olive oil, sesame oil, soybean oil, sunflower oil, fish oil, canola oil, castor oil, corn oil, and mixtures thereof. According to a preferable embodiment, the pharmaceutically acceptable oil is selected from olive oil and fish oil.

Content of the nanosized API in the pharmaceutically acceptable oil is typically from 1 mg/ml to 500 mg/ml, such as from 10 mg/ml to 250 mg/ml, or from 1 mg/ml to 100 mg/ml. If the suspension comprises more than one API, overall content of the APIs in the pharmaceutically acceptable oil is typically from 1 mg/ml to 500 mg/ml.

According to one embodiment, the composition comprises the suspension and one or more pharmaceutically acceptable excipients, such as permeability enhancers, enzyme inhibitors, surfactants, encapsulating agents and coating agents. The surfactant can be anionic, cationic, nonionic, and amphoteric (zwitterionic).

According to one embodiment, the composition comprises a coating agent or an encapsulating agent. The coating is preferable since it protects the API e.g., from acidic environment and enzymatic degradation of said composition in the stomach of a subject. An exemplary coating is an enteric coating, i.e., a polymer that prevents dissolution or disintegration of the API in the gastric environment.

According to a particular embodiment, at least one, preferably all of the pharmaceutically acceptable excipients, if present, are nanosized.

According to one embodiment, the excipients are solid substances dispersed in the oil.

According to another embodiment, the excipients are dissolved in the oil. For example, a surfactant Tween 80, is dissolved in the oil. In practice, a suspension can have several liquid phases and several solid phases, but at least two different phases. Accordingly, the minimum requirement is that suspension comprises one continuous liquid phase comprising the pharmaceutically acceptable oil and one discrete solid phase comprising the nanosized API.

According to a preferable embodiment, the composition does not include a surfactant since surfactants increases the release rate of the nanosized APIs from the suspension to surrounding aqueous environment.

According to another aspect, the present disclosure concerns a composition of the present disclosure for use as a medicament.

According to one embodiment, the API of the composition is nanosized insulin, and the composition is for use in the treatment of diabetes.

According to another embodiment, the API of the composition is nanosized parathyroid hormone (PTH), and the composition is for use in the treatment of hypoparathyroidism.

According to still another embodiment, API of the composition is nanosized oxytocin, and the composition is for use in the start or strengthening of uterine contractions during labor.

According to still another embodiment, the API of the composition is nanosized calcitonin, and the composition is for use in treatment of osteoporosis.

According to still another embodiment, the API of the composition is nanosized vasopressin, and the composition is for use in treatment of low blood pressure.

When the nanosized API is suspended to the pharmaceutically acceptable oil, its release to aqueous environment is hindered compared to the corresponding bulk API suspended in the pharmaceutically acceptable oil.

Manufacture of the nanosized APIs of desired particle size can be done by any procedures known in the art. An exemplary method is disclosed in WO2022112270. The nanosized API may be crystalline, amorphous or their mixtures. If the composition comprises nanosized API and further nanosized ingredients such as excipients, the further ingredients can be nanosized together with API, i.e., mixed with the API prior to nanosizing, or separately.

For manufacturing the composition, a desired amount of nanosized API is mixed with a desired amount of pharmaceutically acceptable oil to form a suspension. Then, the desired amount of the suspension is contacted to one or more pharmaceutically acceptable excipients.

### Experimental

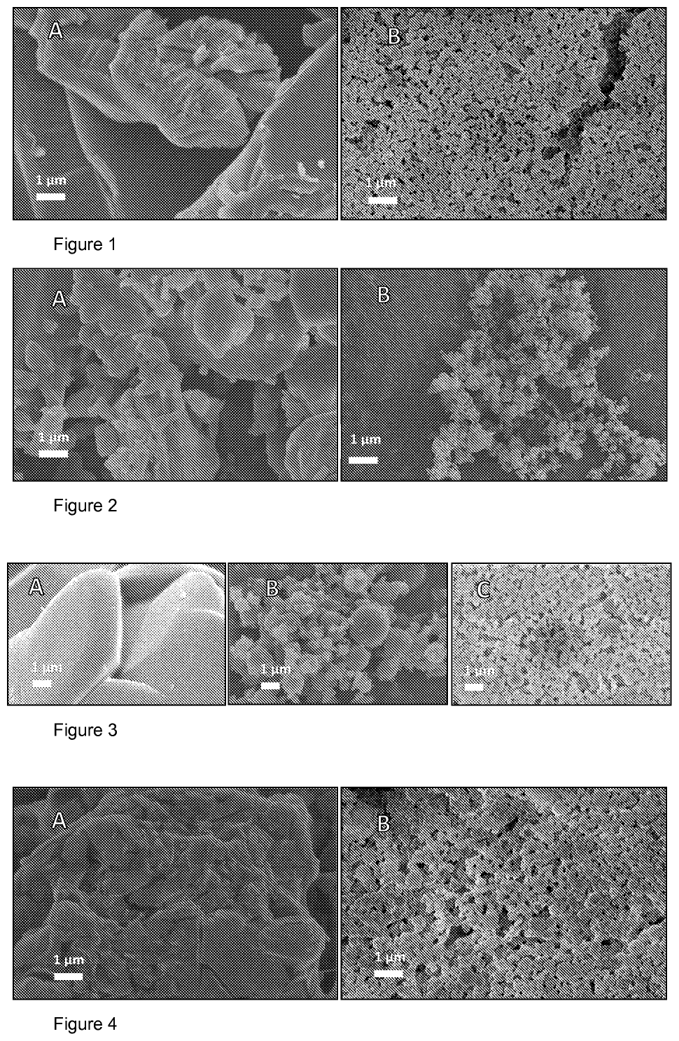

Bulk APIs were nanosized and microsized in house using methods known in the art such as the method disclosed in WO2022112270. API particle size were verified by Scanning Electron Microscope (SEM). Exemplary SEM images of bulk HSA (FIG. 1A), nanosized HSA (FIG. 1B), bulk insulin (FIG. 2A), nanosized insulin (FIG. 2B), bulk lysozyme (FIG. 3A), microsized lysozyme (FIG. 3B), nanosized lysozyme (FIG. 3C), bulk D-mannitol (FIG. 4A) and nanosized D-mannitol (FIG. 4B) are shown in FIGS. 1 to 4, with scale bars of 1 μm.

### Stability of Nanosized Insulin Suspension in a Pharmaceutically Acceptable Oil

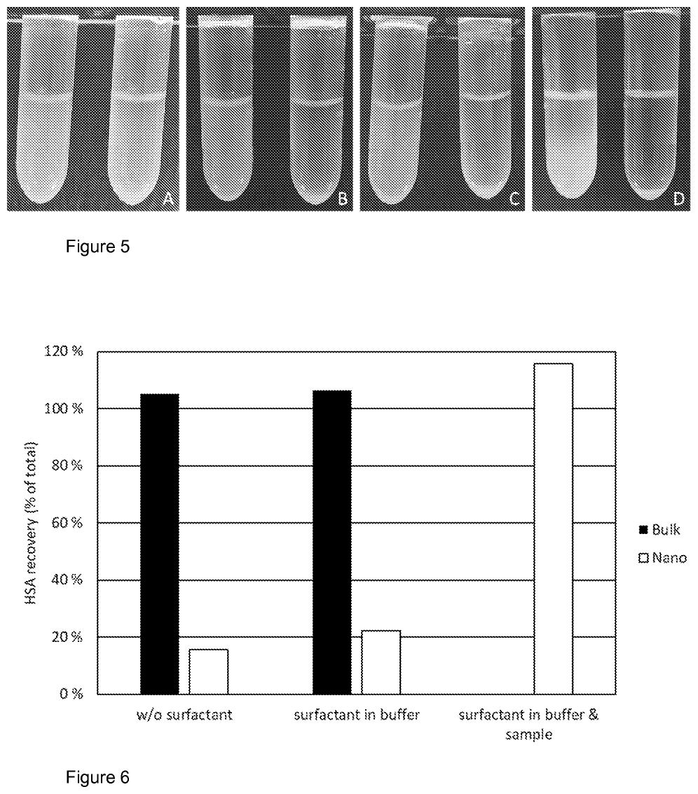

Nanosized insulin or bulk insulin (10 mg, Sigma) was added to 1 ml of fish oil, mixed by vortexing and allowed to stand at room temperature. Results are shown in FIG. 5. As seen from the figure, sedimentation of the nanosized insulin (left tube at each time point) was significantly slower than sedimentation of bulk insulin (right tube at each time point) at t=0 (A), t=30 min (B), t=60 min (C) and t=24 h (D).

Extraction Recovery of Bulk and Nanosized HSA from a Pharmaceutically Acceptable Oil

HSA (Sigma) was added at 10 mg/ml to fish oil as bulk powder or nanosized powder manufactured from said bulk HSA. The suspensions were stored at 2-8° C., for 1 to 4 days. The suspensions were mixed, before analysis, by vortexing. Then, ca. 1 ml of the oil-particle suspension was extracted with 50 ml of 50 mM potassium phosphate buffer (pH 6.8). The extraction was carried out for 1 hour, at 37° C. in a glass flask with continuous mixing using a magnetic stirrer (400 rpm). Certain samples were prepared with Tween 80 in the oil (2% w/w) referred to as “surfactant in sample”. Furthermore, certain samples were extracted using a buffer supplemented with Tween 80 (0.1% w/w). After mixing for 1 hour at 37° C., the mixing was stopped, and the oil phase was allowed to separate from the buffer at room temperature in a so-called 30 min separation of phase. After said separation phase, an oil-free sample of the buffer was taken. Then the HSA concentration of the extracted oil-free sample was measured using Bradford reagent (BioRad) and multiplate reader (Victor Nivo, PerkinElmer). Total recovery was set as amount of HSA (added to oil) per volume of extraction buffer. Each data point was obtained by repeating the dissolution experiment three times. A summary of the results is shown in FIG. 6. As seen in the figure, bulk HSA (black bars) was completely recovered, whereas the recovery of nanosized HSA (white bars) was extremely low in comparison. The nanosized HSA could be recovered from the oil if surfactant was added to the buffer and to the oil.

Extraction Recovery of Bulk and Nanosized Insulin from a Pharmaceutically Acceptable Oil

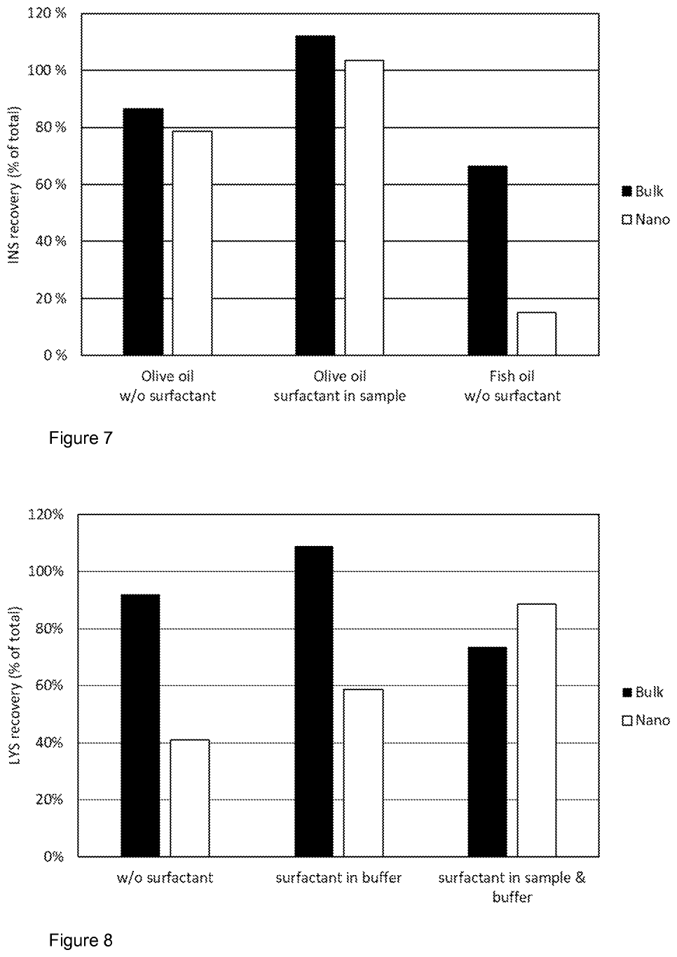

Insulin (Sigma) was added at 10 mg/ml to olive oil or fish oil as bulk powder or nanosized powder manufactured from said bulk insulin. The suspensions were stored at 2-8° C. for 1 to 4 days. The suspensions were mixed, before analysis, by vortexing. Then, ca. 1 ml of the oil-particle suspension was extracted with 50 ml of 50 mM potassium phosphate buffer (pH 6.8) for 1 hour, at 37° C. in a glass flask with continuous mixing using a magnetic stirrer (400 rpm). Certain samples were prepared with Tween 80 in the oil (2% w/w) referred to as “surfactant in sample”. After mixing for 1 hour at 37° C. the mixing was stopped, and the oil phase was allowed to separate from the buffer at room temperature in a so-called 30 min separation of phase. After said separation phase, an oil-free sample of the buffer was taken. Then the insulin concentration of the extracted oil-free sample was measured using an Ultra-High-Performance Liquid Chromatography (UHPLC) method (based on Ph.Eur monograph). Total recovery was set as amount of insulin (added to oil) per volume of extraction buffer. Each data point was obtained by repeating the dissolution experiment three times. A summary of the results is shown in FIG. 7. As seen in the figure, recovery of nanosized insulin (white bars) from fish oil was less efficient compared to the recovery of bulk insulin (black bars) from said oil. In contrast, from olive oil both nanosized and bulk insulin could be recovered as efficiently. Furthermore, adding surfactant to the olive oil suspensions, the insulin recovery was improved further. It is apparent that the extraction of the APIs is dependent on the quality of the pharmaceutically acceptable oil.

Extraction Recovery of Bulk and Nanoformed Lysozyme from a Pharmaceutically Acceptable Oil

Lysozyme (from chicken egg, Sigma) was added at 10 mg/ml to fish oil as bulk powder or nanosized powder manufactured from said bulk lysozyme. After storing at +4° C. over-night or for 3 days and mixing by vortexing, 0.5 ml of the oil was extracted with 25 ml of 50 mM potassium phosphate buffer, pH 6.8 (sample w/o surfactant) for 1 h, at 37° C. in a glass flask with continuous mixing using a magnetic stirrer (350 rpm). For samples with surfactant in buffer, 0.1% (w/v) Tween 80 was added to the buffer before extraction. For samples with surfactant in sample and buffer, Tween 80 was added to the oil (2% w/w) and 0.1% (w/v) Tween 80 was added to the buffer before extraction. After a 30 min separation of phases at room temperature, the protein concentration of the buffer was measured with Bradford reagent (BioRad) and multiplate reader (Victor Nivo, PerkinElmer). For baseline levels and standard curves, pure fish oil was extracted with the same extraction method (FOEB). For protein content determinations FOEB was used both as a blank, and for the lysozyme based standard curve samples (0-0.4 mg/ml). Total recovery was set as total amount of lysozyme (added to oil) per volume of extraction buffer. Each data point was obtained by repeating the assay three times, each time with three parallels of each sample. A summary of the results is shown in FIG. 8. As seen from the figure, nanosized lysozyme (white bars) could be recovered from the oil only by addition of surfactant to the buffer and to the oil, whereas bulk lysozyme (black bars) could be extracted without surfactant.

### Comparative Example. Extraction Recovery of Bulk and Nanosized D-Mannitol from a Pharmaceutically Acceptable Oil

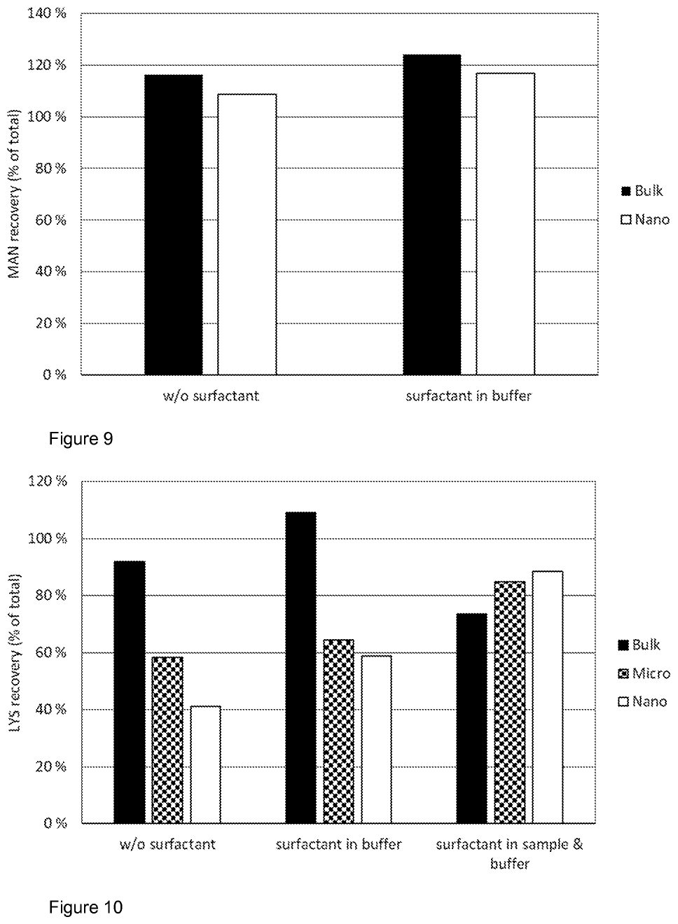

D-mannitol (Sigma) was added at 10 mg/ml to olive oil as bulk powder or nanosized powder manufactured from said bulk powder. After mixing by vortexing and storing at +4° C. over-night or for 3 days, around 1 ml of the oil was extracted with 50 ml of 50 mM potassium phosphate buffer, pH 6.8 for 1 h, at 37° C. in a glass flask with continuous mixing using a magnetic stirrer (400 rpm). Part of the samples were prepared with Tween 80 in the oil (2% w/w). After a 30 min separation of phases at room temperature, the mannitol concentration of the buffer was measured with a D-Mannitol Colorimetric Assay Kit (Sigma) and a multiplate reader (Victor Nivo, PerkinElmer). Total recovery was set as amount of mannitol (added to oil) per volume of extraction buffer. Each data point was obtained by repeating the assay twice, each time with two parallels of each sample. A summary of the results is shown in FIG. 9. As seen from the figure, bulk D-mannitol (black bars) and nanosized D-mannitol (white bars) can be equally well recovered from olive oil.

Effect of Particle Size on Extraction Recovery from a Pharmaceutically Acceptable Oil

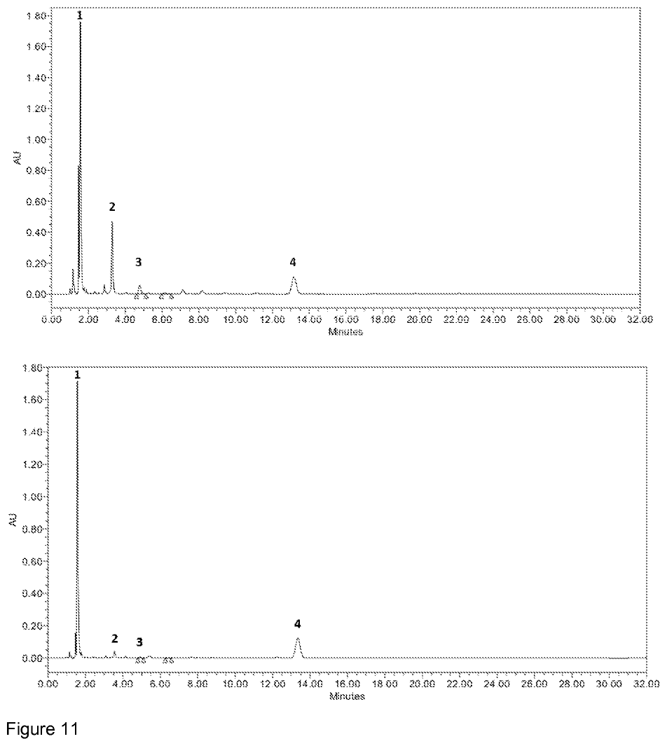

Lysozyme (from chicken egg, Sigma) was added at 10 mg/ml to fish oil as bulk powder, microsized powder or nanosized powder. After storing at +4° C. over-night or for 3 days and mixing by vortexing, 0.5 ml of the oil was extracted with 25 ml of 50 mM potassium phosphate buffer, pH 6.8 (sample w/o surfactant) for 1 h, at 37° C. in a glass flask with continuous mixing using a magnetic stirrer (350 rpm). For samples with surfactant in buffer 0.1% (w/v) Tween 80 was added to the buffer before extraction. For samples with surfactant in sample and buffer, Tween 80 was added to the oil (2% w/w) and 0.1% (w/v) Tween 80 was added to the buffer before extraction. After a 30 min separation of phases at room temperature, the protein concentration of the buffer was measured with Bradford reagent (BioRad) and a multiplate reader (Victor Nivo, PerkinElmer). For baseline levels and standard curves, pure fish oil was extracted with the same extraction method (FOEB). For protein content determinations FOEB was used both as a blank, and for the lysozyme based standard curve samples (0-0.4 mg/ml). Total recovery was set as total amount of lysozyme (added to oil) per volume of extraction buffer. Each data point was obtained by repeating the assay three times, each time with three parallels of each sample. A summary of the results is shown in FIG. 10. As seen from the figure, in the absence of surfactant, recovery of the nanosized lysozyme (white bars) was less efficient than recovery of the bulk lysozyme (black bars) or the microsized lysozyme (checkered bars).

### Differential Protection Against Proteolytic Degradation of Bulk and Nanosized Insulin in Oil.

Insulin (Sigma) was mixed into fish oil either as bulk or nanosized insulin at 10 mg/ml and stored at +4° C. over-night. The following day, 1 ml of the suspension was vortexed and extracted with 50 ml of 50 mM potassium phosphate buffer pH 6.8 and chymotrypsin (0.01 mg/ml, Sigma C4129) for 1 h at 37° C. in a glass flask with continuous mixing using a magnetic stirrer (400 rpm). After the chymotrypsin treatment, 1 mM PMSF (Sigma 93482) and 0.1% (w/v) Tween 80 were added and samples were withdrawn from the buffer phase for UPLC-analyses using Waters ACQUITY PREMIER UPLC, with an ACQUITY Premier CSH C18 1.7 um 2.1×100 mm column. The mobile phases were (A) 20 mM Na₂SO₄ buffer pH 2.3: acetonitrile (82:18) and (B) 20 mM Na₂SO₄ buffer pH 2.3: acetonitrile (50:50) and the flow rate was 0.208 ml/min. Background peaks from oil extracted buffer (1), insulin degradation products (2) undigested insulin (3), PMSF inhibitor (4). Results are shown in FIG. 11. As seen from FIG. 11, bulk insulin (top panel) was released from the oil resulting in proteolytic digestion whereas nanosized insulin (bottom panel) remained protected from digestion.

### Observations

The present invention has one or more of the following advantages
Since the nanosized API is mixed with a pharmaceutically acceptable oil, the dissolution and thus bioavailability of the API released from the oil can be controlled.
Regarding manufacturing, the nanosized APIs are readily dispersed in the pharmaceutically acceptable oils having a slower sedimentation rate, thus they have better colloidal stability, compared to larger API particles.
The suspension protects the nanosized proteins from action of proteases.

1. A composition including a suspension comprising
nanosized particles of one or more active pharmaceutical ingredients (APIs) selected from proteins and peptides, and
one or more pharmaceutically acceptable oils.

2. The composition according to claim 1 wherein molecular weight of the one or more APIs is 500 kDa or less.

3. The composition according to claim 1, wherein the one or more pharmaceutically acceptable oils are selected from a group consisting of soybean oil, olive oil, fish oil, sesame oil, canola oil, sunflower oil, castor oil, and corn oil.

4. The composition according to claim 1, wherein the one or more pharmaceutically acceptable oils are selected from olive oil and fish oil.

5. The composition according to claim 1, wherein content of the one or more APIs in the one or more pharmaceutically acceptable oil in the suspension is from 1 mg/ml to 500 mg/ml.

6. The composition according to claim 1, wherein the one or more APIs is selected from a group consisting of insulin, parathyroid hormone, oxytocin, calcitonin, and vasopressin.

7. The composition according to claim 1, provided that the composition does not include a surfactant.

8. The composition according to claim 1, comprising one or more pharmaceutically acceptable excipients.

9. The composition according to claim 8 wherein the one or more pharmaceutically acceptable excipients are selected from a group consisting of permeability enhancers, enzyme inhibitors, surfactants, encapsulating agents, and coating agents.

10. The composition according to claim 8, wherein at least one, preferably all of the pharmaceutically acceptable excipients are nanosized.

11. The composition according to a claim 1, comprising a coating that inhibits degradation of the one or more APIs in an acidic medium.

12. The composition according to claim 11 wherein the coating is an enteric coating.

13. The composition according to a claim 1, consisting of a suspension consisting of the nanosized particles of one or more active pharmaceutical ingredients (APIs) selected from proteins and peptides, and one or more pharmaceutically acceptable oils.

14. The composition according to claim 1, comprising a suspension consisting of the nanosized particles of one or more active pharmaceutical ingredients (APIs) selected from proteins and peptides, and one or more pharmaceutically acceptable oils.

15. The composition according to claim 1, for use as a medicament.
